(19) United States
(12) Patent Application Publication
(10) Pub. No.: US 2016/0146779 A1
(43) Pub. Date: May 26, 2016

(54) PUMPLESS BREATH ANALYSIS SYSTEM

(71) Applicant: Breathometer, Inc., Burlingame, CA (US)

(72) Inventors: Jonathan Gallagher, San Francisco, CA (US); Kenton Ngo, San Francisco, CA (US); Likang Xue, Santa Clara, CA (US); Silpesh Patel, Brisbane, CA (US); Tim Ratto, Milbrae, CA (US)

(21) Appl. No.: 14/943,777

(22) Filed: Nov. 17, 2015

Related U.S. Application Data
(60) Provisional application No. 62/083,132, filed on Nov. 21, 2014.

(51) Int. Cl. G01N 33/497

(52) U.S. Cl. CPC G01N 33/4972

(57) ABSTRACT

A breath analysis device has a housing that limits pressure from a user's breath that passes over a sensor, improving sensor readings of the sensor. The housing includes an inlet opening, one or more outlet openings, and an inner cavity. The inner cavity defines a path between the inlet opening and the one or more outlet openings. The inner cavity includes a sensor housing, a high resistance path between the inlet opening and the sensor housing, a first low resistance path between the housing and a first subset of outlet openings of the one or more outlet openings, and a second low resistance path between the inlet opening and a second subset of outlet openings of the one or more outlet openings. Breath that passes into the sensor housing is analyzed by sensors in the sensor housing.

### CROSS-REFERENCE TO RELATED APPLICATIONS

This application claims the benefit of U.S. Provisional Application No. 62/083,132, filed Nov. 21, 2014, which is incorporated by reference in its entirety.

### BACKGROUND

1. Field of the Disclosure

The present disclosure relates to a gas sensors and more specifically to a portable breathalyzer devices for measuring a blood alcohol content (BAC) in a user.

2. Description of the Related Art

Breathalyzers are devices for estimating the blood alcohol content (BAC) from a breath sample of a user. Typical breathalyzers include gas sensors that generate an electrical signal when alcohol is present in the breath sample of the user. Gas sensors used to analyze the concentration of alcohol in the breath sample are sensitive to variations in the pressure of the sample passing through the sensor. In addition, to obtain measurements that are better correlated with the amount of alcohol in the user's blood, a breath sample that originates from the deep lung of the user, where partitioning of volatile organic chemicals (VOCs) from blood into breath is most reproducible, is desired.

To address these problems, breathalyzers use pumps to draw breath samples into a sensor chamber. The pump may start drawing air into the sensor chamber after a set time delay (e.g., after a 5 second delay). The addition of a pump to a breathalyzer increases the size and the power consumption of the breathalyzer.

### SUMMARY

A breath analysis device determines a concentration of an analyte present in the breath of a user by controlling the pressure of the breath over a sensor. For instance, the breath analysis device may determine the concentration of ethanol in the breath of a user. The determined concentration of ethanol in the breath of the user may be used to estimate the amount of alcohol in the user's blood.

The breath analysis device includes a housing and a sensor. The housing includes an inlet opening, one or more outlet openings, and an inner cavity. The inner cavity defines a path between the inlet opening and the one or more outlet openings. The inner cavity includes a sensor housing, a high resistance path between the inlet opening and the sensor housing, a first low resistance path between the housing and a first subset of outlet openings of the one or more outlet openings, and a second low resistance path between the inlet opening and a second subset of outlet openings of the one or more outlet openings.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates the operating architecture of a breath analysis system for analyzing different compounds, such as alcohol, in the breath of a user, according to one embodiment.

FIG. 2A illustrates a top view of a pumpless breath analysis device, according to one embodiment.

FIG. 2B illustrates a cross-sectional side view of the pumpless breath analysis device, according to one embodiment.

FIG. 3A illustrates a sample breath flow inside the pumpless breath analysis device, according to one embodiment.

FIG. 3B illustrates a box diagram of a sample breath flow inside the pumpless breath analysis device, according to one embodiment.

FIG. 4 illustrates a user interface for providing instructions to a user during the analysis of the user's breath, according to one embodiment.

FIG. 5 illustrates a user interface for providing the analysis results, according to one embodiment.

FIG. 6 is a block diagram illustrating components of an example machine able to read instructions from a machine-readable medium and execute them in a processor (or controller).

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Breath Analysis Operating Architecture

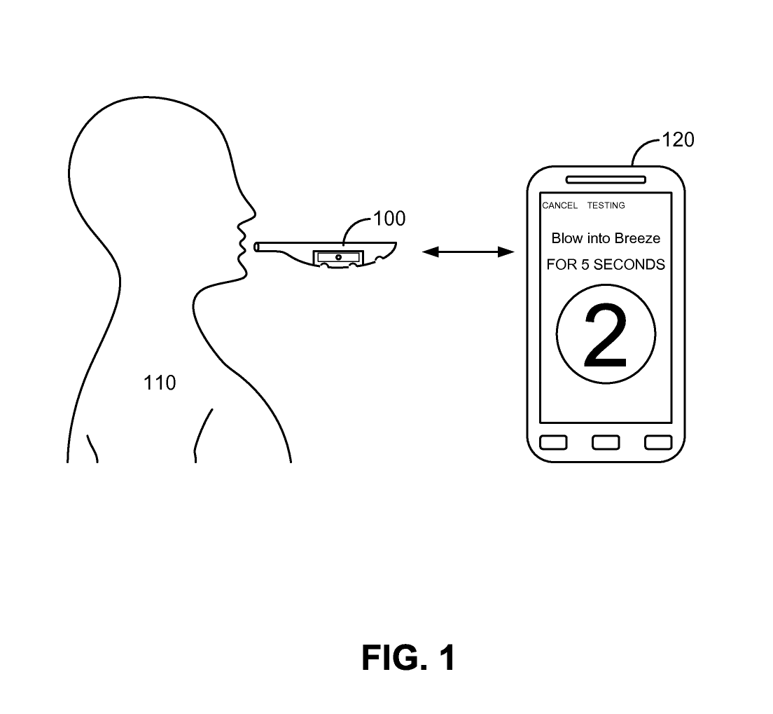

FIG. 1 illustrates an operating architecture of a breath analysis system for analyzing different compounds, such as alcohol, in the breath of a user, according to one embodiment. The breath analysis system includes a breath analysis device 100, such as a pumpless breath analysis device, a user 110 using the breath analysis device 100, and a client device 120 connected to the breath analysis device 100. In some embodiments, the client device 120 is a handheld computing device, such as a smartphone. The client device 120 may connect to the breath analysis device via a wired connection or wirelessly (e.g., via Bluetooth).

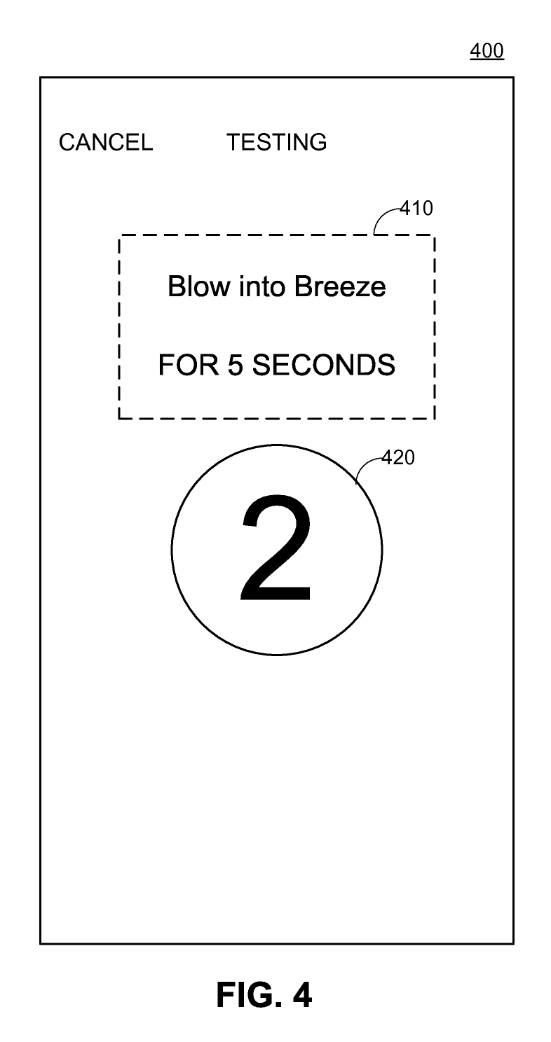

The client device 120 may receive an indication from the user to start the analysis. In some embodiments, the client device 120 provides instructions to the user 110 for performing the analysis with the breath analysis device 100. For instance, the client device 120 may instruct the user to blow into the breath analysis device for a predetermined amount of time (e.g. 5 seconds). The client device 120 may additionally display a countdown of the number of seconds left to complete de analysis. The client device 120 may also initialize the breath analysis device 100 prior to instructing the user. A more detailed description of the user interface displayed by the client device is provided in conjunction with FIG. 4.

In some embodiments, the client device 120 may connect with a server (not shown). The server may store results of the breath analysis from several devices and keep track of the performance of the breath analysis devices and may recalibrate the breath analysis devices 100 periodically. The server may also associate certain devices with specific manufacturing conditions, such as a specific lot, manufacturing version, or timeframe. The server may keep track of a drift in the measured alcohol concentration in a user's breath for breath analysis devices of a specific lot to identify that the devices associated with the lot need recalibration. The server identifies client devices 120 connected to a breath analysis device 100 associated with the specific lot and sends an update of calibration parameters. The calibration parameters may be applied by the mobile device 120 or may update settings on the breath analysis device 100. In some embodiments, the server pushes an update when a shift in the results of the analysis is larger than a threshold value. In other embodiments, the server pushes the update periodically (e.g., every 6 months).

### Pumpless Sampling for Breath Analysis Devices

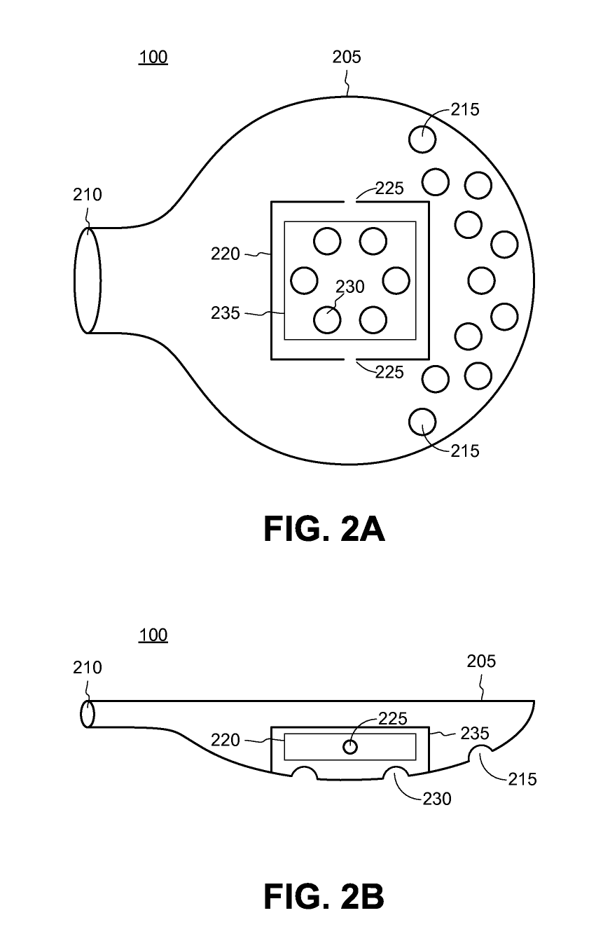

FIG. 2A illustrates a top view of a pumpless breath analysis device and FIG. 2B illustrates a cross-sectional side view of the pumpless breath analysis device, according to one embodiment. The pumpless breath analysis device 100 permits effective sampling of breath without a pump while still controlling the analysis of controlling parameters of the breath passing the sensor for a deep lung exhale. The pumpless breath analysis device 100 includes a housing 205, a mouthpiece 210, one or more outlet openings or holes 215,230, a sensor housing 220, one or more sensor inlet holes 225, and a gas sensor 235. The pumpless breath analysis device may include additional components such as a battery, a controller module, and/or a wireless transceiver.

The housing 205 houses the internal component of the pumpless breath analysis device. At one end of the housing 205, the pumpless breath analysis device includes a mouthpiece 210. The mouthpiece 210 includes an inlet opening to receive a breath sample from a user. The housing additionally includes one or more outlet holes 215, 230 to release the breath sample that entered the housing 205 through the mouthpiece 210. The outlet holes may be sensor outlet holes 230 for releasing the breath sample that entered the sensor housing 220, or housing outlet holes 215 for releasing the breath sample that entered the housing 205 but did not enter the sensor housing 220. The outlet holes 215, 230 may be 0.5 mm to 5 mm in diameter. The housing 205 may include 1 to 50 outlet holes 215, 230. In some embodiments, the number and size of the outlet holes 215, 230 are based on the amount of air to be released from the pumpless breath analysis device. For instance, the size and number of outlets holes 215, 230 may be directly proportional to the size of the inlet opening of the mouthpiece 210. That is, the larger the inlet opening of the mouthpiece 210, the larger the number of outlet holes 215, 230 and/or the size of each of the outlet holes 215, 230. In some embodiments, the total opening area of the outlet holes 215, 203, that is, the aggregated area of each of the outlet holes 215, 203, is smaller than the total opening area of the inlet opening of the mouthpiece 210. In particular, the number and size of outlet holes 215,230 may vary the resistance of air to leaving the housing.

The sensor housing 220 houses the gas sensor 235. The gas sensor 235, for instance, measures the concentration of volatile organic chemicals (VOCs), such as ethanol, in the breath sample. The sensor housing 220 includes one or more sensor inlet holes 225. The sensor inlet holes 225 may be 0.5 mm to 5 mm in diameter, and the sensor housing 220 may include 1 to 20 sensor inlet holes 225. In some embodiments, the sensor inlet holes 225 are smaller than the outlet holes 215, 230. The sensor inlet holes 225 may be position so that the sensor inlet holes are not facing the mouthpiece 210. For instance, the sensor inlet holes 225 are positioned at an angle greater than 45° with respect to the mouthpiece 210. In some embodiments, the sensor inlet holes 225 are positioned at an angle greater than 90° with respect to the mouthpiece 210. Alternatively, the sensor inlet holes 225 are located all around the sensor housing 220. The size and number of sensor inlet holes 225 may vary the resistance of a sample entering the sensor housing and thereby passing over the gas sensor 235.

The housing 205 further includes one or more sensor outlet holes 230. The sensor outlet holes 230 provide an exit path from the housing 205 for the breath sample that entered the sensor housing 220 through the sensor inlet holes 225. The sensor outlet holes 230 may be 0.5 mm to 5 mm in diameter, and the housing 205 may include 1 to 20 sensor outlet holes 230. In some embodiments, the size of the sensor outlet holes 230 is larger than the size of the sensor inlet holes 225. The size of the sensor outlet holes 230 may be substantially equal to the size of the housing outlet holes 215.

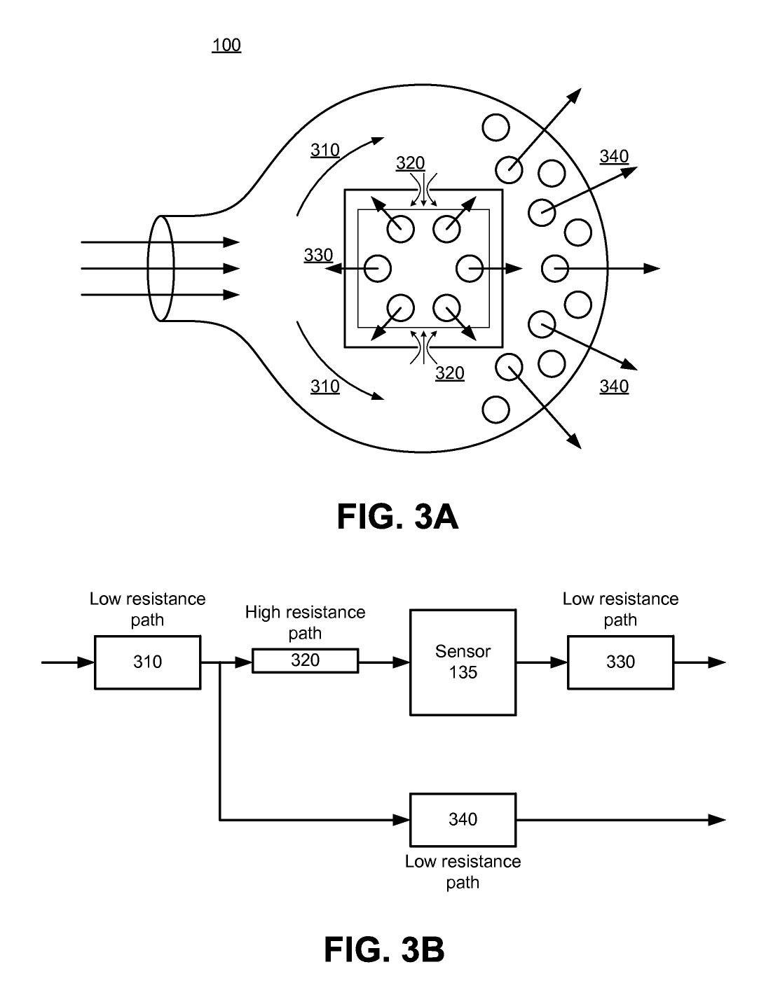

FIG. 3A illustrates a sample breath flow inside the pumpless breath analysis device 100, and FIG. 3B illustrates a box diagram of the sample breath flow inside the pumpless breath analysis device 100, according to one embodiment. The breath sample enters the pumpless breath analysis device 210 via the mouthpiece 210. The breath sample travels through the housing 205 and enters the sensor housing 220 by following based on air paths created by the resistance of the various housing outlet holes 215, sensor inlet holes 225, and sensor outlet holes 230. The housing 205 forms a first low resistance path 310 for the breath sample to flow through. The breath sample can then either enter the sensor housing 220 through the sensor inlet holes 225 or exit the housing 205 through the housing outlet holes 215. A high resistance path 320 is provided to the sensor housing 220 while a low resistance path 340 is provided to exit the housing 205. Since the resistance provided by the number and size of sensor inlet holes 225 is higher than the number and size of housing outlet holes 215, the sensor inlet holes 225 form the high resistance path 320 for the breath sample to flow through, and the outlet holes 225 form the low resistance path 340 for the breath sample to flow through. As a result, a larger amount of the breath sample travels through the housing 205 and exits the housing 205 via the housing outlet holes 215 relative to the portion of the breath sample that passes into the sensor housing 220.

A smaller amount of breath sample enters the sensor housing 220 via the sensor inlet hole 225 through the high resistance path 320. The sample is analyzed by the gas sensor 235, and exits the pumpless breath analysis device 100 via the sensor outlet holes 230. Since the resistance provided by the number and size of sensor outlet holes 230 is larger than the resistance provided by the number and size, the sensor outlet holes form a low resistance path 330 for the breath sample that entered the sensor housing to exit the pumpless breath analysis device 100. As a result, while there is a high resistance path to enter the sensor housing, the low-resistance path reduces the pressure over the gas sensor 235 relative to the pressure at which the sample passes into the sensor housing.

In some embodiments, since the sensor outlet holes 330 form a low resistance path for breath to exit the sensor housing, the inside of the sensor housing is kept at a relatively constant pressure during the breath sampling. This may further improve the accuracy of the measurement performed by the gas sensor 235.

### User Interface for Breath Analysis System

FIG. 4 illustrates an exemplary user interface 400 for providing instructions to a user during the analysis of the user's breath, according to one embodiment. The user interface 400 shows the user 110 instructions 410 on the steps to perform during the analysis of the user's breath. For instance, the user interface 400 of FIG. 4 is instructing the user to blow into the breath analysis device 100 for 5 second. Additionally, the user interface 400 includes a countdown of the number of seconds left for the analysis. The exemplary user interface 400 of FIG. 4 instructs the user to blow into the breath analysis device 100 for 2 more seconds. In some embodiments, the countdown starts when the breath analysis device 100 detects that the user has started blowing into the breath analysis device 100.

The breath analysis device 100 may information to the client device 120 while a breath sample is being provided. This information may indicate, for example, fluctuations in the breath sample, sensor readings, or other information. The client device 120 in some embodiments provides additional instructions may to the user based on this information, for example to blow harder or lighter into the breath analysis device 100, or not to block the outlet holes.

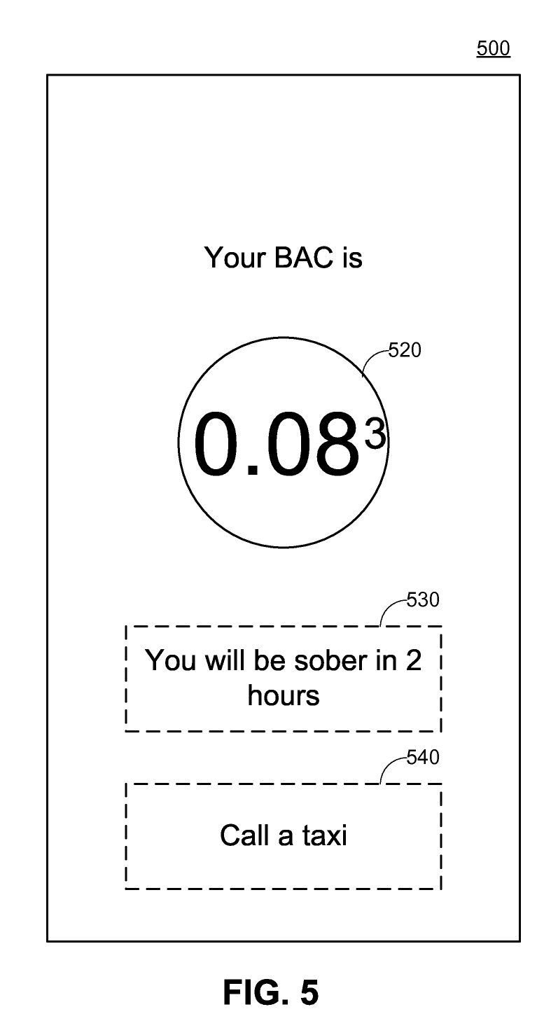

FIG. 5 illustrates an exemplary user interface 500 for providing the analysis results, according to one embodiment. The user interface 500 includes a graphical element 520 that shows the blood alcohol content (BAC) of the user 110. In the exemplary user interface, the BAC of the user is 0.083%. The user interface 500 may display additional information 530 such as an amount of time that a user's BAC is expected to return to 0.000%.

In some embodiments, client device determines suggestions for the user based on the BAC of the user. The user interface 500 displays the suggestions 540 to the user based on the results of the analysis. For instance, if the user has a BAC that is greater than a legal driving limit, the user interface may suggest the user to call a taxi. In some embodiments, the suggestion is an interface to perform the suggested task. For instance, selecting graphical element 540 may initiate a call to a taxi provider. The client device 120 may additionally send the taxi provider with the current location of the user and the address of the user's home. Other suggestions include contacting a nearby friend, or searching for a nearby hotel.

In some embodiment, the client device 120 may activate or deactivate certain functionality of the client device 120 based on the results of the analysis. For instance, if the BAC of a user is larger than a threshold, the client device 120 may not allow the user to call or text phone numbers included in a “do not drunk call” list. In another example, the client device 120 may not allow the user access online shopping apps or sites, or access the user's online bank account. The client device 120 may additionally block applications on the client device 120 from accessing certain content on the client device 120, such as pictures or video, or may prevent applications from sending pictures of video. In some embodiments, the client device automatically notifies a second user (e.g., the user's wife) of the BAC and the location of the user, if the BAC of the user is higher than a threshold.

### Computing Machine Architecture

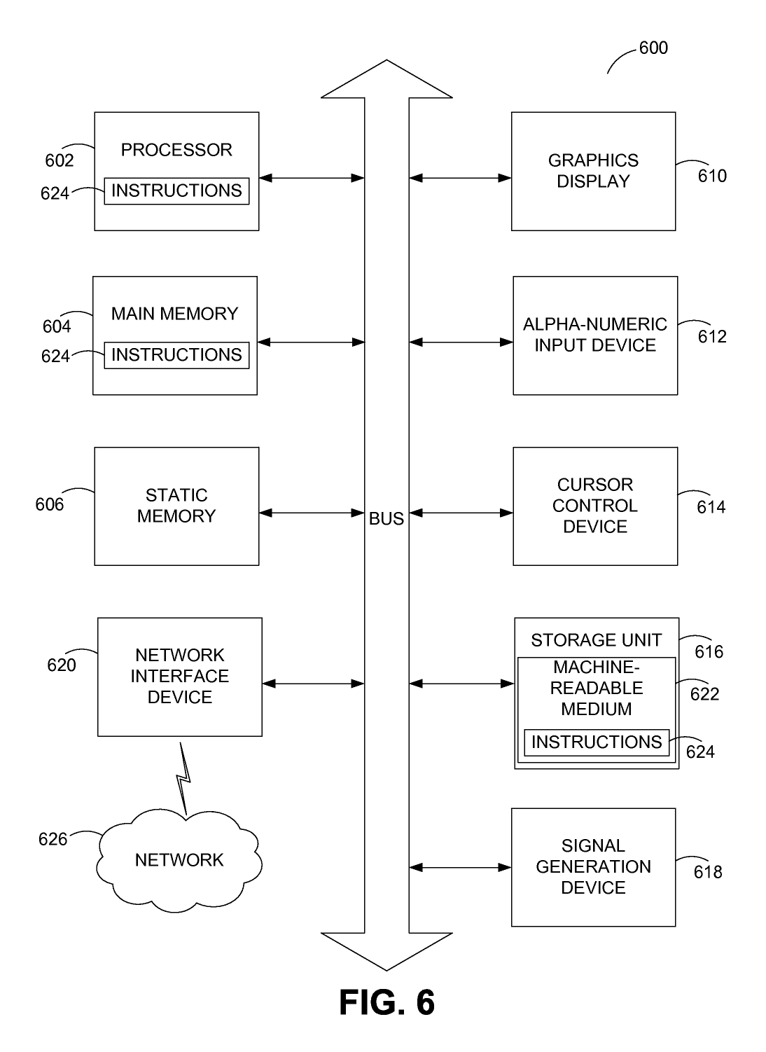

FIG. 6 is a block diagram illustrating components of an example machine able to read instructions from a machine-readable medium and execute them in a processor (or controller). Specifically, FIG. 6 shows a diagrammatic representation of a machine in the example form of a computer system 600 within which instructions 624 (e.g., software) for causing the machine to perform any one or more of the methodologies discussed herein may be executed. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The machine may be a server computer, a client computer, a personal computer (PC), a tablet PC, a set-top box (STB), a personal digital assistant (PDA), a cellular telephone, a smartphone, a web appliance, a network router, switch or bridge, or any machine capable of executing instructions 624 (sequential or otherwise) that specify actions to be taken by that machine. Further, while only a single machine is illustrated, the term “machine” shall also be taken to include any collection of machines that individually or jointly execute instructions 624 to perform any one or more of the methodologies discussed herein.

The example computer system 600 includes a processor 602 (e.g., a central processing unit (CPU), a graphics processing unit (GPU), a digital signal processor (DSP), one or more application specific integrated circuits (ASICs), one or more radio-frequency integrated circuits (RFICs), or any combination of these), a main memory 604, and a static memory 606, which are configured to communicate with each other via a bus 608. The computer system 600 may further include graphics display unit 610 (e.g., a plasma display panel (PDP), a liquid crystal display (LCD), a projector, or a cathode ray tube (CRT)). The computer system 600 may also include alphanumeric input device 612 (e.g., a keyboard), a cursor control device 614 (e.g., a mouse, a trackball, a joystick, a motion sensor, or other pointing instrument), a storage unit 616, a signal generation device 618 (e.g., a speaker), and a network interface device 820, which also are configured to communicate via the bus 608.

The storage unit 616 includes a machine-readable medium 622 on which is stored instructions 624 (e.g., software) embodying any one or more of the methodologies or functions described herein. The instructions 624 (e.g., software) may also reside, completely or at least partially, within the main memory 604 or within the processor 602 (e.g., within a processor's cache memory) during execution thereof by the computer system 600, the main memory 604 and the processor 602 also constituting machine-readable media. The instructions 624 (e.g., software) may be transmitted or received over a network 626 via the network interface device 620.

While machine-readable medium 622 is shown in an example embodiment to be a single medium, the term “machine-readable medium” should be taken to include a single medium or multiple media (e.g., a centralized or distributed database, or associated caches and servers) able to store instructions (e.g., instructions 624). The term “machine-readable medium” shall also be taken to include any medium that is capable of storing instructions (e.g., instructions 624) for execution by the machine and that cause the machine to perform any one or more of the methodologies disclosed herein. The term “machine-readable medium” includes, but not be limited to, data repositories in the form of solid-state memories, optical media, and magnetic media.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

What is claimed is:

1. A breath analysis device comprising:
a housing having an inlet opening, one or more outlet openings, and an inner cavity, the inner cavity defining a path between the inlet opening and the one or more outlet openings, the inner cavity including:
a sensor housing,
a high resistance path between the inlet opening and the sensor housing,
a first low resistance path between the sensor housing and a first subset of outlet openings of the one or more outlet openings, and
a second low resistance path between the inlet opening and a second subset of outlet openings of the one or more outlet openings; and
a sensor disposed inside the sensor housing of the inner cavity of the housing, the sensor configured to analyze a concentration of an analyte in a gas sample.

2. The breath analysis device of claim 1, wherein the high resistance path between the inlet opening and the sensor housing comprises:
one or more inlet openings in the sensor housing, wherein the one or more inlet openings in the sensor housing are smaller than the one or more outlet openings of the housing.

3. The breath analysis device of claim 2, wherein the one or more inlet openings in the sensor housing have a diameter between 0.5 mm and 5 mm.

4. The breath analysis device of claim 2, wherein the sensor housing has between 1 and 20 inlet openings.

5. The breath analysis device of claim 2, wherein the one or more inlet openings in the sensor housing are not facing the inlet hole of the housing.

6. The breath analysis device of claim 2, wherein a total opening area of the inlet openings in the sensor housing is smaller than a total opening area of the one or more outlet openings of the housing.

7. The breath analysis device of claim 2, wherein a total opening area of the inlet openings in the sensor housing is smaller than a total opening area of the outlet openings of the first subset of outlet openings.

8. The breath analysis device of claim 1, further comprising:
a third low resistance path between the inlet opening and the high resistance path.

9. The breath analysis device of claim 1, wherein the sensor disposed inside the sensor housing is configured to measure a concentration of alcohol in a gas sample.

10. The breath analysis device of claim 1, wherein a number and size of the outlet openings are directly proportional to a size of the inlet opening.

11. The breath analysis device of claim 1, wherein the inlet opening comprises a mouthpiece.

12. A breath analysis device comprising:
a housing having:
an inlet opening for receiving a breath sample to analyze,
a plurality of outlet openings for providing an exit path for the breath sample to leave the exit the housing, and
an inner cavity, the inner cavity including a sensor housing, the sensor housing including one or more sensor housing inlet openings, the sensor housing inlet openings having a size smaller than the plurality of outlet openings;
a sensor disposed inside the sensor housing of the inner cavity of the housing.

13. The breath analysis device of claim 12, wherein the sensor housing inlet openings have a diameter between 0.5 mm and 5 mm.

14. The breath analysis device of claim 12, wherein the sensor housing has between 1 and 20 inlet openings.

15. The breath analysis device of claim 12, wherein the one or more sensor housing inlet openings are not facing the inlet hole of the housing.

16. The breath analysis device of claim 12, the sensor housing further comprises a subset of outlet openings of the plurality of outlet openings of the housing.

17. The breath analysis device of claim 12, wherein a first subset of outlet openings of the plurality of outlet openings of the housing are disposed on an outer surface of the sensor housing.

18. The breath analysis device of claim 12, wherein the sensor housing is disposed in a central position inside the inner cavity of housing.

19. The breath analysis device of claim 12, wherein the sensor disposed inside the sensor housing is configured to measure a concentration of alcohol in a gas sample.
